(19) United States
(12) Patent Application Publication
(10) Pub. No.: US 2023/0405208 A1
(43) Pub. Date: Dec. 21, 2023

(54) MOTORIZED SUCTION DILATION AND CURETTAGE DEVICE

(71) Applicant: William Cutler, Austin, TX (US)

(72) Inventor: William Cutler, Austin, TX (US)

(21) Appl. No.: 17/844,354

(22) Filed: Jun. 20, 2022

(51) Int. Cl. A61M 1/00; A61B 17/3207; A61M 29/02

(52) U.S. Cl. CPC A61M 1/842; A61B 17/320708; A61M 1/80; A61M 29/02; A61B 2217/005

(57) ABSTRACT

A motorized suction dilation and curettage device includes a handheld grip with an integrated motor and a tube interface for coupling between the motor and a rotatable cylinder. The rotatable cylinder grips a rotatable tube coupled between a curette and a flexible hose of a vacuum machine. The vacuum machine generates a suction force through the flexible hose, rotatable tube, and attached curette. When the motorized suction dilation and curettage device is activated, the motor generates a rotational force that is applied to the rotatable tube and the attached curette via the tube interface, thereby enabling a health care provider to control motorized rotation of the curette.

### BACKGROUND

### Technical Field

The disclosed embodiments relate to a motorized suction dilation and curettage device.

### Description of Related Art

Suction dilation and curettage is a procedure to remove the product of conception from a woman's uterus. Health care providers often perform this procedure after miscarriages. In a conventional suction dilation and curettage procedure, a surgeon uses one hand to anchor a tenaculum on the anterior lip of the cervix and uses the other hand to maneuver a vacuum curette around the uterus to scrape and vacuum uterine tissue through suction tubing connected to a suction machine. Performing this procedure with conventional instruments is often time-consuming and can cause significant fatigue in the surgeon's hand and wrist.

### BRIEF DESCRIPTION OF THE DRAWINGS

FIG. 1 is an example embodiment of a motorized suction dilation and curettage system.

FIG. 2 is an example embodiment of a tubing assembly for a motorized suction dilation and curettage system.

FIG. 3 illustrates a first embodiment of a handheld motor for a motorized suction dilation and curettage system.

FIG. 4 illustrates a second embodiment of a handheld motor for a motorized suction dilation and curettage system.

FIG. 5 illustrates a bottom perspective view of a tube interface for a motorized suction dilation and curettage system.

FIG. 6 is a first example embodiment of a tube interface for a motorized suction dilation and curettage system having separable upper and lower components.

FIG. 7 is a second example embodiment of a tube interface for a motorized suction dilation and curettage system having separable upper and lower components.

FIG. 8A is a first example embodiment of a tube assembly coupled to a tube interface for a motorized suction dilation and curettage system.

FIG. 8B is a second example embodiment of a tube assembly coupled to a tube interface for a motorized suction dilation and curettage system.

FIG. 9A is an assembled side view of an internal construction of a first example embodiment of a tube interface for a motorized suction dilation and curettage system.

FIG. 9B is a perspective view of a subset of components of a first example embodiment of a tube interface for a motorized suction dilation and curettage system.

FIG. 9C is an exploded view of an internal construction of a first example embodiment of a tube interface for a motorized suction dilation and curettage system.

FIG. 10A is an assembled side view of an internal construction of a second example embodiment of a tube interface for a motorized suction dilation and curettage system.

FIG. 10B is an exploded view of an internal construction of a second example embodiment of a tube interface for a motorized suction dilation and curettage system.

FIG. 11 is another example embodiment of a tube interface for a motorized suction dilation and curettage system.

### DETAILED DESCRIPTION

The Figures (FIGS.) and the following description describe certain embodiments by way of illustration only. One skilled in the art will readily recognize from the following description that alternative embodiments of the structures and methods illustrated herein may be employed without departing from the principles described herein. Reference will now be made to several embodiments, examples of which are illustrated in the accompanying figures. Wherever practicable, similar or like reference numbers may be used in the figures and may indicate similar or like functionality.

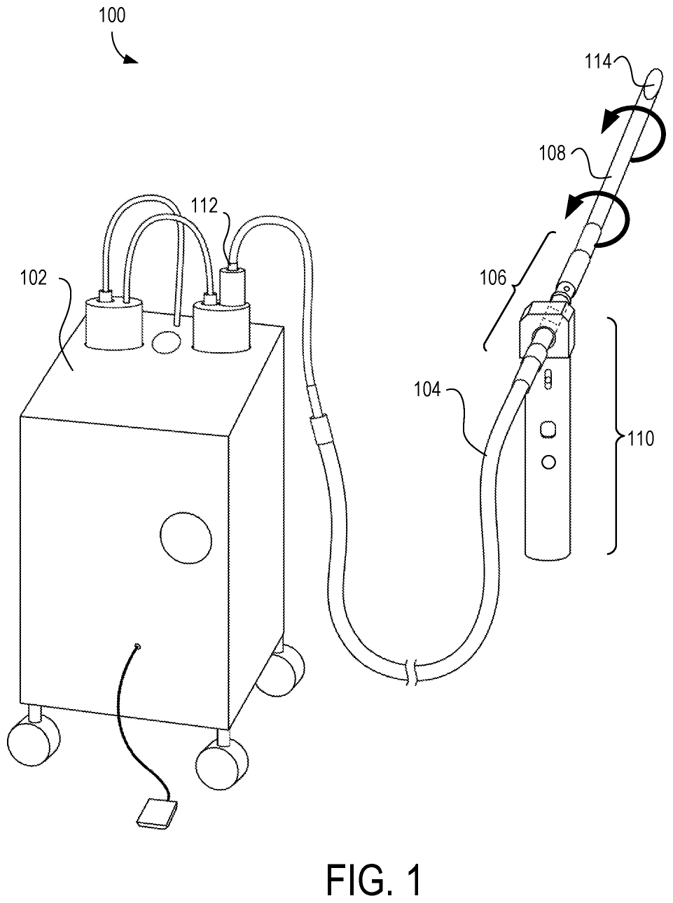

FIG. 1 is an example embodiment of a motorized suction dilation and curettage system 100. The motorized suction dilation and curettage system 100 includes a vacuum machine 102, a flexible hose 104, a rotatable tube 106, a curette 108, and a handheld motor 110. The vacuum machine 102 generates a suction force through one or more nozzles 112 coupled to one end of the flexible hose 104. The opposite end of the flexible hose 104 is attached to the rotatable tube 106 in a manner that allows the rotatable tube 106 to freely rotate about its longitudinal axis relative to the flexible hose 104. In an embodiment, the flexible hose 104 comprises a flexible PVC (polyvinyl chloride) or other flexible plastic. The rotatable tube 106 may comprise a clear rigid PVC, other rigid plastic, or other rigid material. In an embodiment, the diameter of the flexible hose 104 and rotatable tube 106 may be approximately ¼″, ⅜″, ½″, or ⅝″, although any diameter tubing may be used.

The curette 108 comprises a rigid tube with an open tip 114 designed for scraping and suctioning uterine tissue. In an embodiment, the curette 108 comprises a clear rigid PVC, other rigid plastic, or other rigid material. The curette 108 securely attaches to the rotatable tube 106 at an opposite end from the open tip 114 such that a substantially airtight seal is formed and the curette 108 rotates together with the rotatable tube 106. In various embodiments, the curette 108 may comprise a straight tube as illustrated or may have varying curvatures. Furthermore, the curette 108 may be constructed in various lengths and shapes that may be suitable for different applications. When the vacuum machine 102 is active, it applies suction through the flexible hose 104, the rotatable tube 106, and the curette 108 to generate a suction force at the tip 114 of the curette sufficient to suction uterine tissue.

The handheld motor 110 mechanically couples to the rotatable tube 106. When activated, the handheld motor 110 applies a rotational force to the rotatable tube 106 that causes the rotatable tube 106 and the attached curette 108 to rotate about their collective longitudinal axes. The handheld motor 110 may include various buttons and/or status indicators to enable control and feedback associated with operation. For example, during a dilation and curettage procedure, a physician may grasp the handheld motor 110 in a single hand and control motorized rotation of the curette 108 using the buttons. Use of the motorized suction dilation and curettage system 100 may significantly reduce fatigue experienced by the surgeon, reduce duration of the procedure, and allow for more precise control of the curette 108 relative to traditional methods.

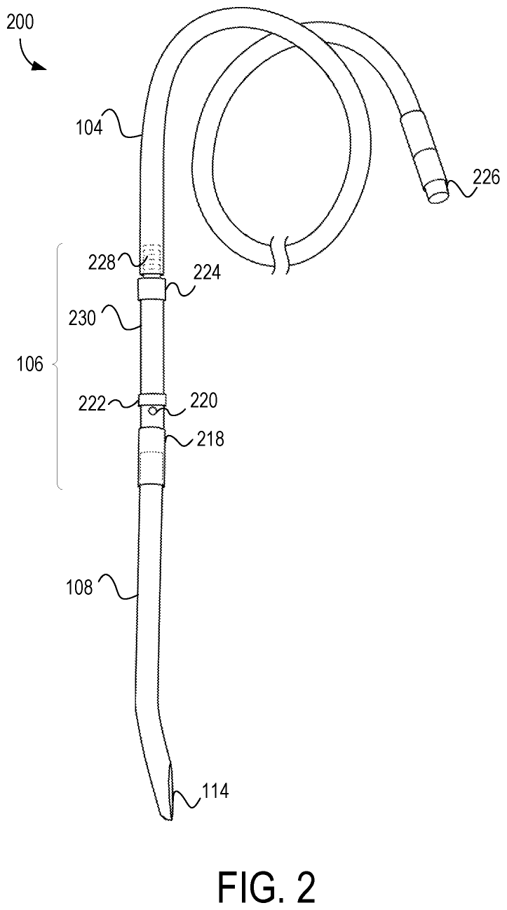

FIG. 2 illustrates an example embodiment of a standalone tubing assembly 200 that is detachable from the handheld motor 110. The tubing assembly 200 comprises the flexible hose 104, the rotatable tube 106, a ribbed compression fitting, 228, and the curette 108 as described above. In this embodiment, the rotatable tube 106 comprises a hose attachment cylinder 224, a main cylinder 230 with a relief hole 220, a slidable hole cover 222, and a curette attachment cylinder 218. The ribbed compression fitting 228 comprises a ribbed cylinder that slides inside an end of the flexible hose 104 at one end and attaches to the rotatable tube 106 at the other end. The hose attachment cylinder 224 of the rotatable tube 106 is rotatably attached to the ribbed compression fitting 228 (i.e., it rotates relative to the compression fitting 228 secured within the flexible hose 104) and is fixedly attached to the main cylinder 230 of the rotatable tube 106 (i.e., the main cylinder 230 and hose attachment cylinder 224 rotate together). The opposite end of the main cylinder 230 comprises a curette attachment cylinder 218 sized to enable the curette 108 to be slidably inserted and substantially secured inside the main cylinder 230. For example, in an embodiment, the inner surface of the main cylinder 230 and/or the outer surface of the curette 108 may have slightly tapered surfaces to enable a secure fit. The curette 108 may be easily removed and re-attached to enable a health care provider to select an appropriate type of curette 108 for a given procedure or quickly switch between them. When assembled, the fitting attachment cylinder 224, main cylinder 230, and curette 108 all rotate together relative to the ribbed compression fitting 228 and flexible hose 104. The hose attachment cylinder 224 and the curette attachment cylinder 218 may have wider outer diameters than the main cylinder 230. Alternatively, the various cylinders 224, 218, 230 may have uniform diameters. In other embodiments, at least one of the hose attachment cylinder 224, curette attachment cylinder 218, and main cylinder 230 may have outer surfaces with non-circular cross-sections (e.g., square, pentagonal, hexagonal, octagonal, etc.)

The relief hole 220 is a small opening in the main cylinder 230 that when uncovered, enables air to escape from the main cylinder 230 thereby significantly reducing the suction force through the curette tip 114. The slidable cover 222 has a diameter slightly wider than the main cylinder 230 and is slidable along the along the main cylinder 230. When positioned over the relief hole 220, the slidable cover 222 blocks the air from escaping from the main cylinder 230, thereby increasing the suction force through the tip 114. The diameters of the fitting attachment cylinder 224 and curette attachment cylinder 218 may be wider than the diameter of the main cylinder 230 to block the slidable cover 222 from sliding off the main cylinder 230.

In alternative embodiments, the rotatable tube 106 may have different mechanisms for coupling with the flexible hose 104 and/or the curette 108. For example, in a different embodiment, the rotatable tube 106 and the curette 108 may comprise a unibody construction. In another embodiment, the curette 108 may directly attach to the flexible hose 104 using an attachment mechanism that allows the curette 108 to rotate freely relatively to the flexible hose 104. In other embodiments, the flexible hose 104 may be compression fit inside the rotatable tube 106 instead of vice versa or the rotatable tube 106 may be compression fit inside the curette 108 instead of vice versa. In yet further embodiment, the flexible hose 104, rotatable tube 106, and curette 108 may be coupled using other attachment techniques, such as screw threads, latching mechanisms, or other connection mechanisms.

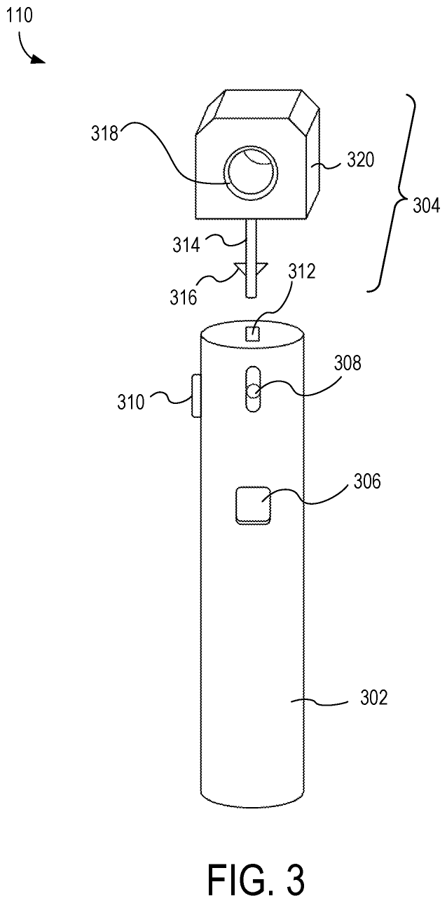

FIG. 3 illustrates an embodiment of a handheld motor 110. The handheld motor comprises a grip 302 and a tube interface 304. The grip 302 is structured to fit conveniently in a physician's hand and may comprise a cylinder or similar form factor. The grip 302 may include an inner cavity (not viewable in FIG. 3) housing internal components such as a battery or wired power interface, a motor, and supporting electronics. The grip 302 may furthermore include various interface controls such as an on/off button 306, a speed control element 308, or other control inputs (e.g., a rotation direction control). The grip 302 may also include a mechanical button 310 or other mechanism for releasing the tube interface 304 from the grip 302 as will be described further below. In an embodiment, the grip 302 may also include various output elements for providing visual, audio, or haptic feedback. For example, the grip 302 may include one or more indicator lights to indicate a power state, a speed setting, whether or not the tube interface 304 is attached, or other information relevant to operation.

The grip 302 includes a slot 312 for coupling with a drive shaft 314 of the tube interface 304. A securing mechanism (e.g., spring-loaded arms 316) may secure the drive shaft 314 to the motor internal to the grip 302 when the drive shaft 314 is inserted into the slot 312 such that the motor applies a rotational force to the drive shaft 314 when activated. For example, the securing mechanism may comprise one or more spring-loaded arms 316 that are pushed in when the drive shaft 314 is inserted into the slot 312 and spring out once inserted to lock the drive shaft 314 in place. The mechanical button 310 depresses the arms 316 when activated to allow the drive shaft 314 to be released from the slot 312. In alternative embodiments, a different coupling mechanism may be utilized to removably couple the tube interface 304 to the grip 302.

The tube interface 304 includes a housing 320 and a rotatable cylinder 318 that is mechanically coupled to the drive shaft 314 (e.g., via a set of gears) such that rotation of the drive shaft 314 causes rotation of the rotatable cylinder 318 relative to the housing 320. In an embodiment, the inner surface of the rotatable cylinder 318 may comprise a compressible gripping material (such as a foam) that grips the outer surface of the rotatable tube 106 to secure the rotatable tube 106 inside the rotatable cylinder 318. In this embodiment, the rotatable tube 106 may be removable from the tube interface 304. In another embodiment, the rotatable tube 106 may be persistently affixed to the inner surface of the rotatable cylinder 318 via an adhesive or other securing mechanism. Alternatively, the rotatable tube 106 and tube interface 304 may comprise a unibody element.

In an alternative embodiment, the drive shaft 314 may be persistently affixed to the internal motor of the grip 302. In this embodiment, the tube interface 304 may include a slot that interfaces with the drive shaft 314 to mechanically couple the rotatably cylinder 318 with the drive shaft 314.

In operation, may use the speed control 308 to control a speed of the motor internal to the grip 302, which in turn controls rotational speed of the drive shaft 314 and the rotatable cylinder 318 of the tube interface 304. The speed control 308 may comprise a set of discrete speed settings (e.g., low, medium, high), or may comprise a continuous range of speeds. In an embodiment, the speed control 308 can control the motor to rotate in either direction (e.g., clockwise or counter-clockwise).

In an example construction, the tube interface 304 may be made of rigid plastic materials intended for single-use operation, i.e., the tube interface 304 may be disposable. The grip 302 may be intended for re-use and may therefore include a housing with more durable materials such as metals, plastics, or a combination thereof. In alternative embodiments, the tube interface 304 and the grip 302 may comprise a unibody construction or may be persistently attached. In an embodiment, a disposable plastic sheet may fit around the grip 302 to prevent it from coming directly in contact with substances during surgical procedures.

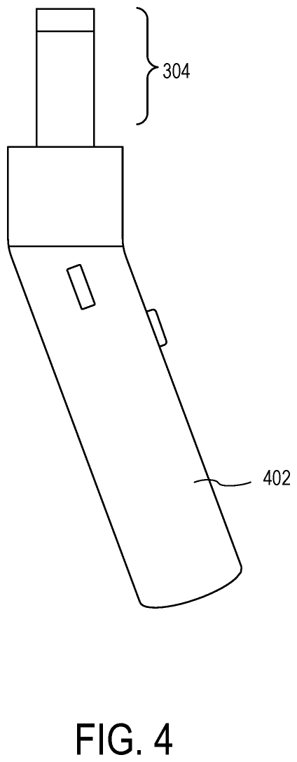

FIG. 4 is a side view of a grip 402 and tube interface 304. The grip 402 operates similarly to the grip 302 described above but in this embodiment has an angled form factor. The angled form factor may be more comfortable for health care providers in some operations. Other form factors for the grip 402 are similarly possible.

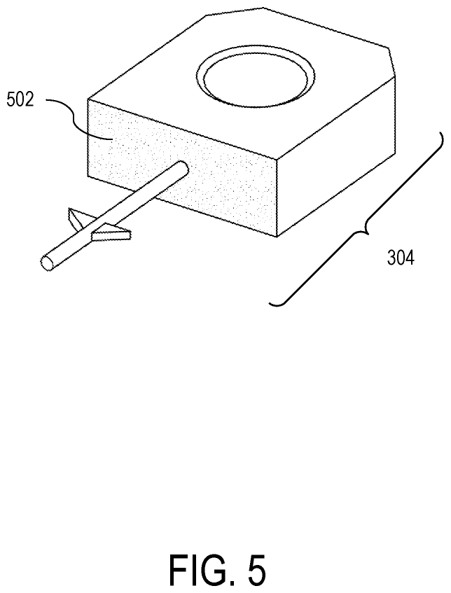

FIG. 5 illustrates a bottom perspective view of the tube interface 304. In this view, a compressible layer 502 is visible that is compressed between the bottom of the tube interface 304 and the top surface of the grip 302 when coupled together, thereby creating a seal that prevents substances from entering the slot 312. The compressible layer may comprise, for example, a compressible foam, silicone, rubber, or other compressible material.

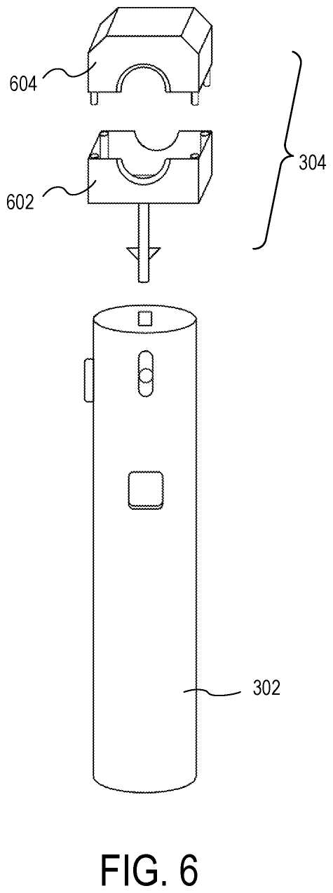

FIG. 6 illustrates an example embodiment of a tube interface 304 that is separable into an upper housing portion 604 and a lower housing portion 602. The upper housing portion 604 may comprise fastening mechanisms that mate with reciprocal fastening mechanisms of the lower housing portion. This embodiment enables the rotatable tube 106 to be inserted into the tube interface 304 when the upper housing portion 604 and the lower housing portion 602 are detached. The rotatable tube 106 is secured within the tube interface 304 when the upper housing portion 604 and the lower housing portion 602 are coupled together. The upper housing portion 604 and the lower housing portion 602 may attach using any suitable fastening mechanisms (e.g., peg-in-hole at each of the four corners, latching mechanisms, screw mechanisms, etc.).

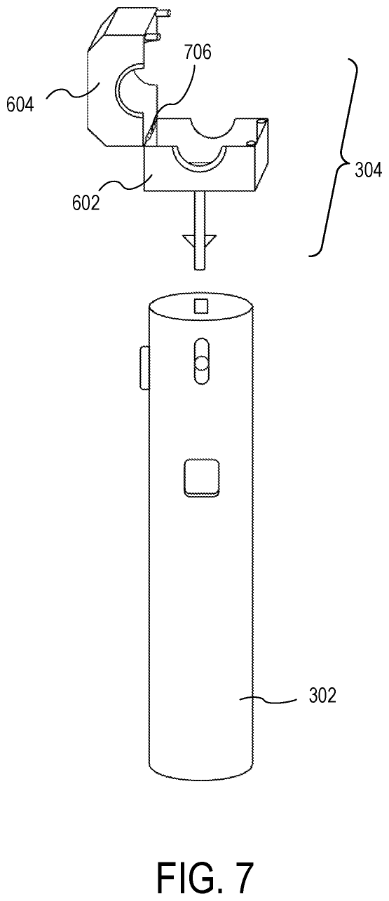

FIG. 7 illustrates another embodiment of a tube interface 304 in which an upper housing portion 604 and a lower housing portion 602 are coupled by a hinge 706 and are therefore not entirely separable. Here, the upper housing portion 604 is pivotable relative to the lower housing portion 602 about the hinge 706 to switch between an open position when the respective fastening mechanisms are decoupled and a closed position when the respective fastening mechanisms are coupled between the upper housing portion 604 and the lower housing portion 602. In this embodiment, the fastening mechanisms may be positioned on the side of the housing opposite the hinge 706.

In other embodiments, the tube interface 304 comprises a housing 320 with a unibody construction that does not separate. In these embodiments, the rotatable tube 106 may be persistently affixed inside the rotatable cylinder 318 of the tube interface 304, or the rotatable 106 may be press fit into an end of the rotatably cylinder 318 as described in further detail below.

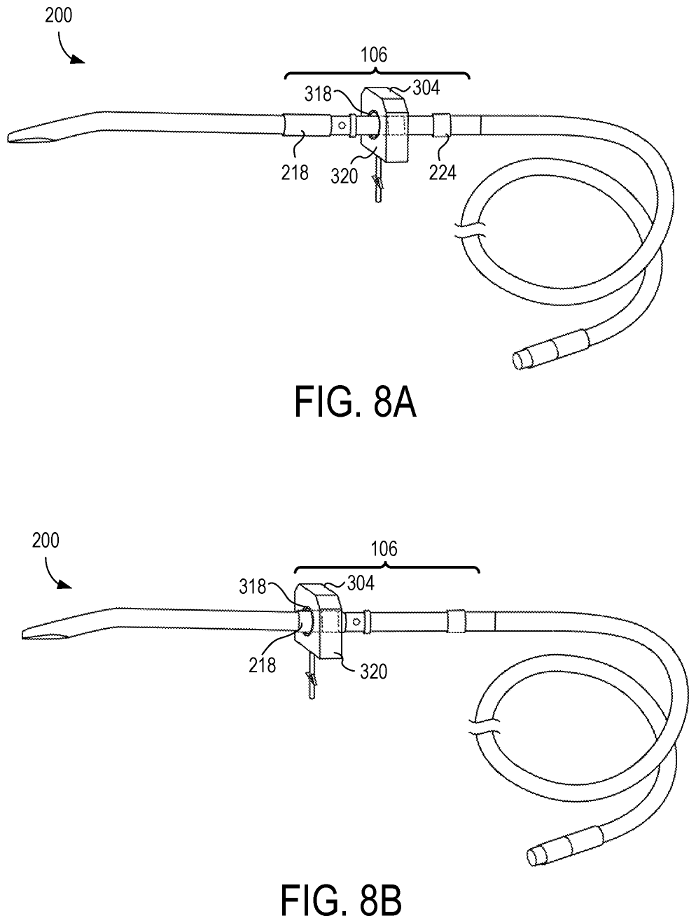

FIGS. 8A-B illustrate various examples of a tube interface 304 coupled with a rotatable tube 106 of a tube assembly 200. In FIG. 8A, the rotatable tube 106 is positioned within the rotatable cylinder 318 in between the curette attachment cylinder 218 and the hose attachment cylinder 224 of the rotatable tube 106. The central portion of the rotatable tube 106 may be persistently affixed inside the rotatable cylinder 318 (e.g., via an adhesive) or the tube interface 304 and rotatable tube 106 may be manufactured as a unibody construction. In alternative embodiments, the housing 320 of the tube interface 304 may be opened up (as described in FIGS. 6-7) to enable the rotatable tube 106 to be inserted or removed in this position. In this embodiment, the rotatable cylinder 318 is sized to have a diameter substantially conforming to the diameter of the main cylinder 230 to securely couple the components together such that the rotatable tube 106 and attached curette 108 will rotate together with the rotatable cylinder 318 when the motor is activated.

In FIG. 8B, the rotatable cylinder 318 fits over the curette attachment cylinder 218. The curette attachment cylinder 218 may be persistently affixed inside the rotatable cylinder 318 (e.g., via an adhesive) or the tube interface 304 and rotatable tube 106 may be manufactured as a unibody construction. Alternatively, the curette attachment cylinder 218 may be press fit into the rotatable cylinder 318 of the tube interface 304. This embodiment enables the rotatable tube 106 to be inserted or removed from the tube interface 304 without the tube interface 304 necessarily having a housing 320 that can open up (as described in FIGS. 6-7). For example, the housing 320 may comprise a unibody construction. In this embodiment, the rotatable cylinder 318 is sized to have a diameter substantially conforming to the diameter of the curette attachment cylinder 218 to securely couple the components together such that the rotatable tube 106 and attached curette 108 will rotate together with the rotatable cylinder 318 when the motor is activated.

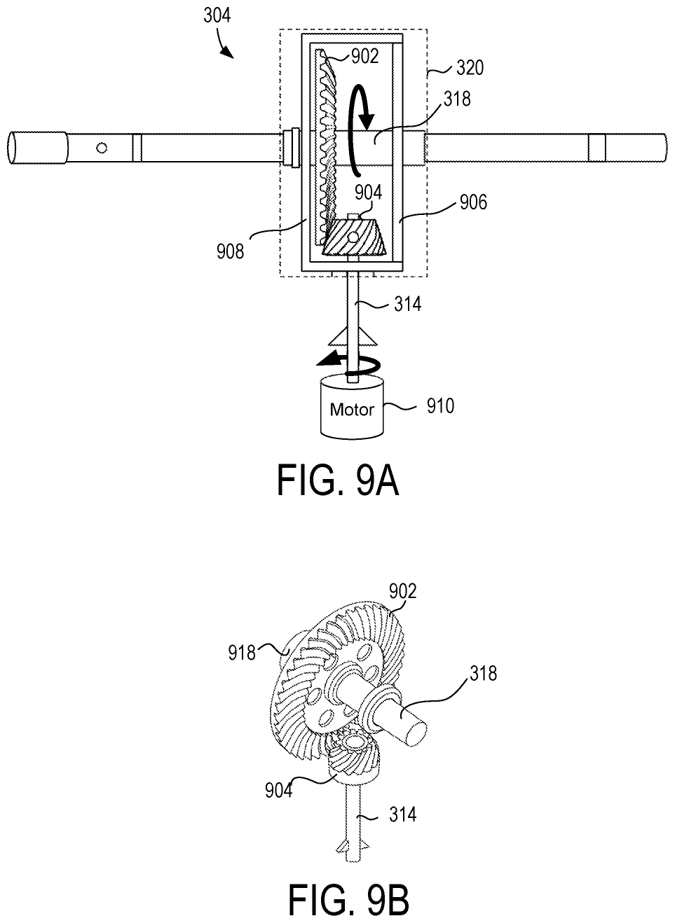

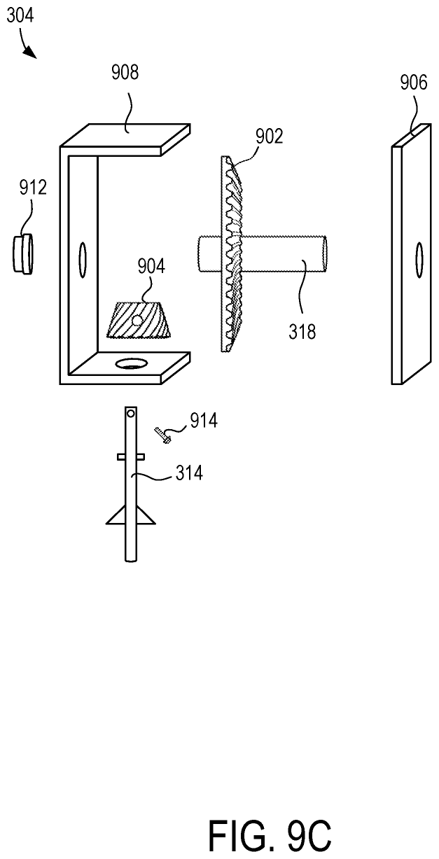

FIGS. 9A-C illustrate an internal construction of the tube interface 304. Particularly, FIG. 9A illustrates a side view of the assembled tube interface 304, FIG. 9B illustrates a perspective view of a subset of the internal components, and FIG. 9C illustrates an exploded view. The tube interface 304 includes a set of gears 902, 904 secured within the housing 320 using a set of brackets 906, 908. The gears 902, 904 are arranged in a right-angle configuration in which a lower gear 904 (coupled to the drive shaft 314) interfaces with an upper gear 902 orientated substantially perpendicular to the lower gear 902. When the motor 910 is activated, it rotates the drive shaft 314 and the lower gear 904. The lower gear 904 drives the upper gear 902 such that the rotational force produced by the motor 910 is translated 90 degrees to the upper gear 902. The upper gear 902 includes or is affixed to the rotatable cylinder 318 that drives the rotatable tube 106. For example, the upper gear 902 and rotatable cylinder 318 may comprise a unibody construction or may be persistently attached (e.g., via an adhesive). The lower gear 904 may be affixed to the drive shaft 314 via a fastener 914 through respective pin holes of the drive shaft 314 and lower gear 904. A set of brackets 906, 908 secure the gears 902, 904 in place. The brackets 906, 908, when assembled, may form a box structure with a hole through a bottom face for the drive shaft 314, and holes on opposite vertical faces to support the rotatable cylinder 318. A securing clip 912 may secure the rotatable cylinder 318 to the bracket 908. While the illustrated embodiment shows a three-sided bracket 908 and a single sided bracket 906, other structural configurations may alternatively be utilized.

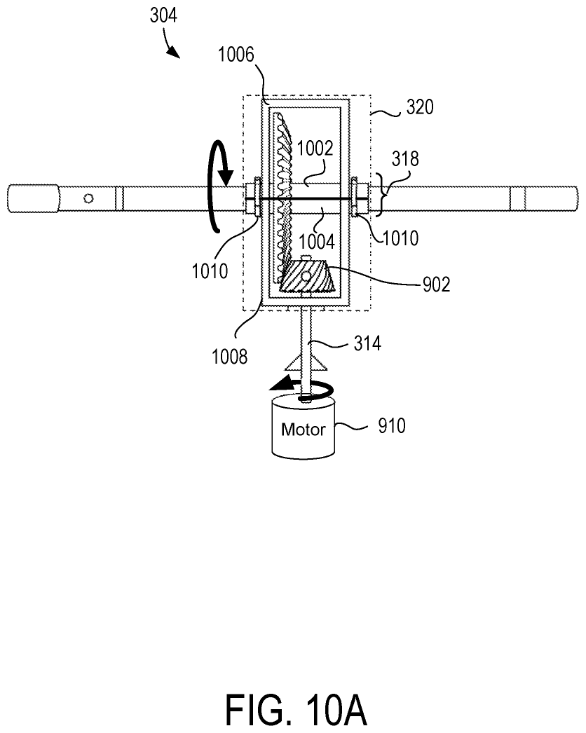

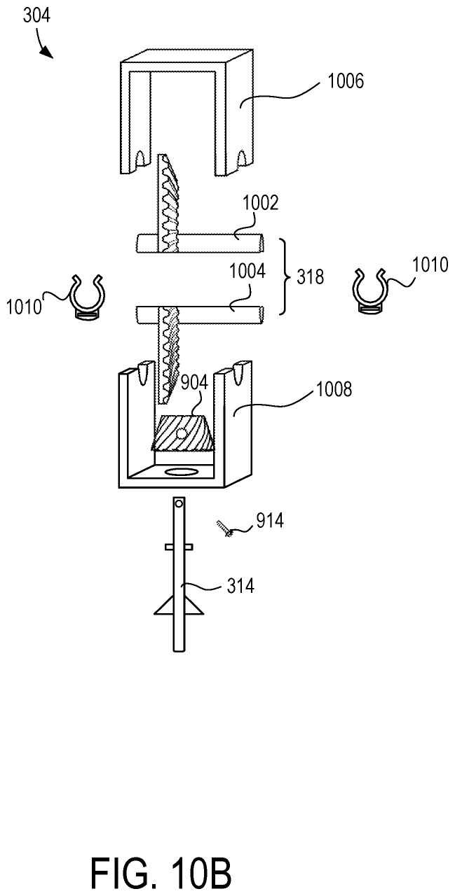

FIGS. 10A-B illustrate an alternative internal construction of a tube interface 304. Particularly, FIG. 10A illustrates a side view of the assembled tube interface 304 and FIG. 10B illustrates an exploded view. In this embodiment, the tube interface 304 can be separated into upper and lower components per the embodiments of FIGS. 6-7 for insertion or removal of the rotatable tube 106. This embodiment includes an upper gear 902 and rotatable cylinder 318 comprising a separable upper gear portion 1002 and a lower gear portion 1004. The upper gear portion 1002 is affixed to an upper bracket 1006 within a top portion of the housing 320 of the tube interface 304. The lower gear portion 1004 is affixed to a lower bracket 1008 within a lower portion of the housing 320 of the tube interface 304. Locking clips 1010 may secure around the ends of the rotatable cylinder 318 outside of the brackets 1006, 1008 to secure the upper portion 1002 and lower portion 1004 of the rotatable cylinder 318 together once the rotatable tube 106 is installed. In an embodiment, the end clips 1010 may be persistently affixed to one of the upper portion 1002 or lower portion 1004 of the rotatable cylinder 318 while being detachable from the other portion. Thus, an upper assembly (including the upper part of the housing 320, the upper bracket 1006, and the upper gear portion 1002) can be separated and resecured to a lower assembly (including the lower part of the housing 320, the lower bracket 1008, and the lower gear portion 1004).

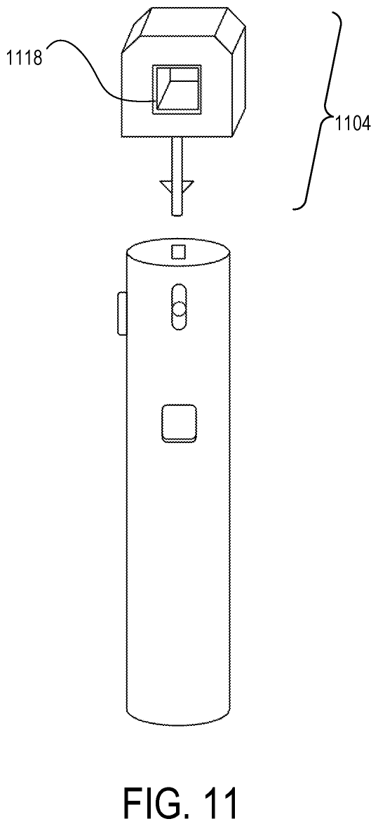

FIG. 11 illustrates another alternative embodiment of a tube interface 1104 that includes a rotatable cylinder 1118 having a square instead of circular base. In this embodiment, the tube interface 1104 may secure to a rotatable tube 106 having a reciprocally structure outer surface with a square cross-section. Other embodiments may include rotatable cylinder 1118 and corresponding rotatable tubes 106 having different cross-sectional shapes such as, for example, triangular, pentagonal, hexagonal, octagonal, or other shapes.

The foregoing description of the embodiments has been presented for the purpose of illustration; it is not intended to be exhaustive or to limit the embodiments to the precise forms disclosed. Persons skilled in the relevant art can appreciate that many modifications and variations are possible in light of the above disclosure.

The language used in the specification has been principally selected for readability and instructional purposes, and it may not have been selected to delineate or circumscribe the inventive subject matter. It is therefore intended that the scope is not limited by this detailed description, but rather by any claims that issue on an application based hereon. Accordingly, the disclosure of the embodiments is intended to be illustrative, but not limiting, of the scope of the invention, which is set forth in the following claims.

1. A motorized suction dilation and curettage device, comprising:
a suction hose including:
a flexible hose for coupling to a suction machine;
a rotatable tube coupled to the flexible hose at a first end and having a curette attachment cylinder at a second end that is rotatable about an longitudinal axis relative to the flexible hose;
a curette for securing to the curette attachment cylinder of the rotatable tube such that the curette rotates with the rotatable tube and such that the suction machine generates a suction force through a tip of the curette when activated;
a handheld grip comprising a motor to generate a rotational force; and
a tube interface coupled to the motor and having a rotatable cylinder that grips the rotatable tube, the tube interface to translate the rotational force of the motor to the rotatable tube to rotate the rotatable tube and the attached curette when the motor is activated.

2. The motorized suction dilation and curettage device of claim 1, wherein the tube interface is persistently attached to the rotatable tube via an adhesive.

3. The motorized suction dilation and curettage device of claim 1, wherein at least a portion of the tube interface and the rotatable tube comprise a unibody construction.

4. The motorized suction dilation and curettage device of claim 1, wherein the tube interface is detachably coupled to the rotatable tube.

5. The motorized suction dilation and curettage device of claim 4, wherein the rotatable cylinder comprises a compressible gripping surface coating an inner surface for compression fitting around the rotatable tube.

6. The motorized suction dilation and curettage device of claim 5, wherein the compressible gripping surface comprises a compressible foam material.

7. The motorized suction dilation and curettage device of claim 4, wherein the rotatable tube is press fit into the rotatable cylinder of the tube interface.

8. The motorized suction dilation and curettage device of claim 1, wherein the tube interface comprises:
a lower housing portion for attaching to the handheld grip, the lower housing portion including a first fastening mechanism and a first portion of the rotatable cylinder;
an upper housing portion including a second fastening mechanism reciprocal to the first fastening mechanism, the upper housing portion attachable to the lower housing portion via a mating between the first and second fastening mechanisms, the upper housing portion including a second portion of the rotatable cylinder; and
wherein the rotatable tube is removable from the tube interface when the first fastening mechanism and the second fastening mechanism are uncoupled from each other, and wherein the rotatable tube is secured to the tube interface within the rotatable cylinder when the first fastening mechanism and the second fastening mechanism are coupled to each other.

9. The motorized suction dilation and curettage device of claim 8, wherein the upper housing portion is completely detached from the lower housing portion when the first fastening mechanism is uncoupled from the second fastening mechanism.

10. The motorized suction dilation and curettage device of claim 8, wherein the tube interface further comprises:
a hinge coupling the lower housing portion and the upper housing portion such that the upper housing portion is pivotable relative to the lower housing portion about the hinge to pivot between an open position when the first fastening mechanism is decoupled from the second fastening mechanism and a closed position when the first fastening mechanism is coupled to the second fastening mechanism.

11. The motorized suction dilation and curettage device of claim 1, wherein tube interface comprises:
a lower gear interfacing with a drive shaft coupled to the motor such that rotation of the drive shaft causes rotation of the lower gear; and
an upper gear coupled to the lower gear such that rotation of the lower gear causes rotation of the upper gear, the upper gear including the rotatable cylinder through a central axis of the upper gear, wherein the upper gear is orientated at a substantially right-angle relative to the lower gear, and wherein the lower gear and the upper gear operate to translate the rotational force of the motor by approximately 90 degrees between the drive shaft and the rotatable tube.

12. The motorized suction dilation and curettage device of claim 11, wherein the upper gear comprises:
an upper gear portion coupled with an upper portion of a housing of the tube interface; and
a lower gear portion coupled with a lower portion of the housing of the tube interface, wherein the upper gear portion and the lower gear portion are separable to enable the rotatable tube to be inserted or removed, and wherein the upper gear portion and the lower gear portion are secured together to form the rotatable cylinder for securing to the rotatable tube when inserted.

13. A motorized suction dilation and curettage device, comprising:
a handheld grip comprising a motor to generate a rotational force; and
a tube interface coupled to the motor and having a rotatable cylinder for gripping a rotatable tube of a tubing assembly having an attached curette, the tube interface to translate the rotational force of the motor to the rotatable tube to rotate the rotatable tube and the attached curette when the motor is activated.

14. The motorized suction dilation and curettage device of claim 13, wherein the tube interface comprises:
a lower housing portion for attaching to the handheld grip, the lower housing portion including a first fastening mechanism and a first portion of the rotatable cylinder;
an upper housing portion including a second fastening mechanism reciprocal to the first fastening mechanism, the upper housing portion attachable to the lower housing portion via a mating between the first and second fastening mechanisms, the upper housing portion including a second portion of the rotatable cylinder; and
wherein the rotatable tube is removable from the tube interface when the first fastening mechanism and the second fastening mechanism are uncoupled from each other, and wherein the rotatable tube is secured to the tube interface within the rotatable cylinder when the first fastening mechanism and the second fastening mechanism are coupled to each other.

15. The motorized suction dilation and curettage device of claim 13, wherein tube interface comprises:
a lower gear interfacing with a drive shaft coupled to the motor such that rotation of the drive shaft causes rotation of the lower gear; and
an upper gear coupled to the lower gear such that rotation of the lower gear causes rotation of the upper gear, the upper gear including the rotatable cylinder through a central axis of the upper gear, wherein the upper gear is orientated at a substantially right-angle relative to the lower gear, and wherein the lower gear and the upper gear operate to translate the rotational force of the motor by approximately 90 degrees between the drive shaft and the rotatable tube.

16. The motorized suction dilation and curettage device of claim 15, wherein the upper gear comprises:
an upper gear portion coupled with an upper portion of a housing of the tube interface; and
a lower gear portion coupled with a lower portion of the housing of the tube interface, wherein the upper gear portion and the lower gear portion are separable to enable the rotatable tube to be inserted or removed, and wherein the upper gear portion and the lower gear portion are secured together to form the rotatable cylinder for securing to the rotatable tube when inserted.

17. A suction tube assembly for a motorized suction dilation and curettage device, the suction tube comprising:
a flexible hose for coupling to a suction machine;
a rotatable tube coupled to the flexible hose such that the suction machine generates a suction force through the rotatable tube, the rotatable tube rotatable about an longitudinal axis relative to the flexible hose, the rotatable tube having a curette attachment cylinder for attaching to a curette; and
a tube interface for coupling to a motor of a handheld grip, the tube interface having a rotatable cylinder that grips the rotatable tube, the tube interface to translate the rotational force of the motor to the rotatable tube to rotate the rotatable tube when the motor is activated.

18. The suction tube assembly of claim 17, wherein the tube interface comprises:
a lower housing portion for attaching to the handheld grip, the lower housing portion including a first fastening mechanism and a first portion of the rotatable cylinder;
an upper housing portion including a second fastening mechanism reciprocal to the first fastening mechanism, the upper housing portion attachable to the lower housing portion via a mating between the first and second fastening mechanisms, the upper housing portion including a second portion of the rotatable cylinder; and
wherein the rotatable tube is removable from the tube interface when the first fastening mechanism and the second fastening mechanism are uncoupled from each other, and wherein the rotatable tube is secured to the tube interface within the rotatable cylinder when the first fastening mechanism and the second fastening mechanism are coupled to each other.

19. The suction tube assembly of claim 17, wherein tube interface comprises:
a lower gear interfacing with a drive shaft coupled to the motor such that rotation of the drive shaft causes rotation of the lower gear; and
an upper gear coupled to the lower gear such that rotation of the lower gear causes rotation of the upper gear, the upper gear including the rotatable cylinder through a central axis of the upper gear, wherein the upper gear is orientated at a substantially right-angle relative to the lower gear, and wherein the lower gear and the upper gear operate to translate the rotational force of the motor by approximately 90 degrees between the drive shaft and the rotatable tube.

20. The suction tube assembly of claim 19, wherein the upper gear comprises:
an upper gear portion coupled with an upper portion of a housing of the tube interface; and
a lower gear portion coupled with a lower portion of the housing of the tube interface, wherein the upper gear portion and the lower gear portion are separable to enable the rotatable tube to be inserted or removed, and wherein the upper gear portion and the lower gear portion are secured together to form the rotatable cylinder for securing to the rotatable tube when inserted.
